(19) United States
(12) Plant Patent Application Publication
(10) Pub. No.: US 2003/0226184 P1
(43) Pub. Date: Dec. 4, 2003

(54) GAURA LINDHEIMERI PLANT NAMED 'BENSO'

(76) Inventor: Howard Bentley, Victoria (AU)

Correspondence Address: CHRISTIE, PARKER & HALE, LLP, 350 WEST COLORADO BOULEVARD, SUITE 500, PASADENA, CA, 91105, US

(21) Appl. No.: 10/160,976

(22) Filed: May 30, 2002

(51) Int. Cl. A01H 5/00

(52) U.S. Cl. PLT/226

(57) ABSTRACT

Described is a new Gaura Lindheimeri plant having pale pink flowers and a dense plant habit.

### BACKGROUND OF THE INVENTION

The present invention relates to a new Gaura Lindheimeri plant having the varietal denomination ‘BENSO.’ ‘BENSO’ was discovered as a seedling variant of the variety Gaura Lindheimeri ‘SISKIYOU PINK’ in a controlled planting.

### SUMMARY OF THE INVENTION

The new variety of a Gaura Lindheimeri plant is an evergreen medium perennial shrub which has a dense, spreading, growing habit distinguished by its pale pink flower color and dense plant habit. Asexual reproduction of the new variety by vegetative cuttings in Wonga Park, Victoria, Australia has demonstrated that the distinguishing characteristics are transferred through succeeding generations.

### COMPARISON WITH PARENT

The new variety differs from its parent, ‘SISKIYOU PINK’, in having a pale pink flower color and a more dense plant habit.

### BRIEF DESCRIPTION OF ILLUSTRATION

The accompanying illustration depicts specimen plants of the new variety in color as true as possible in color photographic illustrations of this nature.

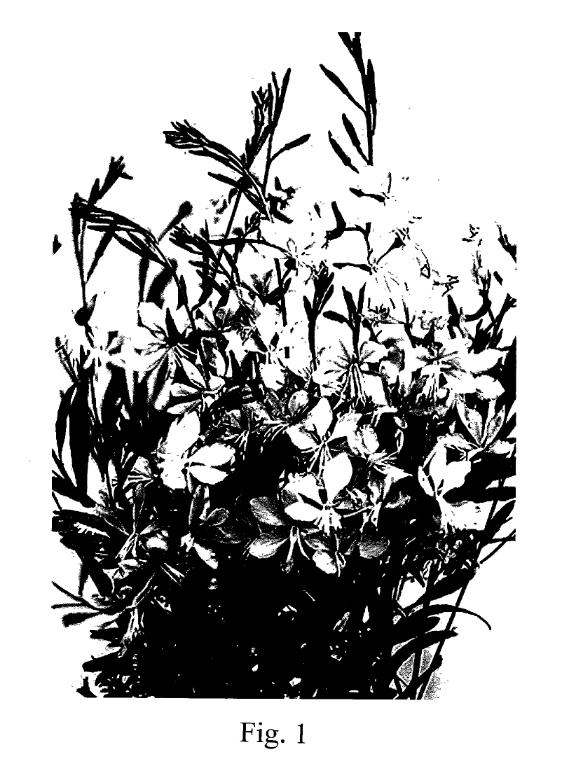

FIG. 1 shows the plant of the new variety.

### DESCRIPTION OF THE NEW VARIETY

The following is a description of the new variety taken from plants grown out-of-doors in Wonga Park, Victoria, Australia during the month of April. Color designations used herein are from the 1995 Royal Horticultural Society Color Chart (“RHSC”).

### PLANT

The plant grows in a spreading habit with dense, vigorous growth, attaining medium height.

### FOLIAGE

Leaflet:

Size.—Medium.

Quantity.—Medium to dense.

Color:

Upper side.—Near 137B.

Under side.—Near 137B.

Shape: Leaves are linear to lanceolate.

Texture:

Upper side.—Smooth with fine pubsescents.

Under side.—Smooth with fine pubsescents.

Veination: Reticulate.

Edge: Slight undulate margins observed.

### WOOD

New shoots:

Color.—Near 137C, 137D and 138A.

Bark.—Smooth with fine pubsescents.

Old wood:

Color.—Near 199A.

Bark.—Smooth with fine pubsescents.

Winter hardiness: Winter hardy to above −5° C.

Resistance to disease: Good, observed to be generally disease resistant.

### REPRODUCTIVE ORGANS

Arrangement: Stamens, filaments and anthers are regularly arranged at the base, but bunched in a pendular formation.

Filaments: White.

Anthers: Crimson to brown.

Pollen: Yellow.

Styles: Thin, singular, long length.

Stigma color Pale green/yellow.

Hips:

Shape.—Oval to linear.

Size.—Small with no seed protrusion.

### FLOWER

Arrangement: Racemes are pendular and of a long length.

Color: Flower bud tip color and base color is near 179B; petal background color is pale pink, near 56D, with petal veins and margins of color red, near 55B.

I claim:

1. A new Gaura Lindheimeri plant of the variety substantially as shown and described.
